(19) United States
(12) Patent Application Publication
(10) Pub. No.: US 2019/0351092 A1
(43) Pub. Date: Nov. 21, 2019

(54) ANTIMICROBIAL HYDROGEL DRESSINGS

(71) Applicant: Argentum Medical, LLC, Geneva, IL (US)

(72) Inventors: Gregg Silver, Chicago, IL (US); Raul Brizuela, Geneva, IL (US)

(21) Appl. No.: 16/323,692

(22) PCT Filed: Aug. 10, 2017
(86) PCT No.: PCT/US17/46281

§ 371 Date: Feb. 6, 2019

Related U.S. Application Data
(60) Provisional application No. 62/372,933, filed on Aug. 10, 2016.

(51) Int. Cl. A61L 15/44; A61F 13/02; A61F 13/00; A61L 15/26; A61L 15/60; A61L 15/42; A61L 15/22; A61L 15/48; A61L 15/18

(52) U.S. Cl. CPC A61L 15/44; A61L 2300/21; A61F 13/0206; A61F 13/0213; A61F 13/00063; A61L 15/26; A61L 15/60; A61L 15/425; A61L 15/225; A61L 15/48; A61L 15/18; A61F 2013/00119; A61F 2013/00285; A61L 2300/104; A61L 2300/404; A61F 13/0243

(57) ABSTRACT

Antimicrobial dressings effective for treating biofilms in wounds are provided. One embodiment provides a wound dressing containing a silver-releasing conformable substrate and hydrogel containing a therapeutic substance. The hydrogel layer optionally may contain a pattern and/or one or more apertures, and may be secured by an optional non-adherent netting. A preferred substrate is a silver-coated substrate, for example silver-coated nylon. The silver-coated nylon can be a knitted, woven, compound, or complex fabric. Silver fibers can be combined within non-woven fabrics. The silver containing substrate, can be non-adherent and/or may contain one or more apertures, and/or may contain elastane. The dressing optionally contains one or more of: an adhesive layer, a separation layer, a moisture regulation layer, a film layer, and combinations thereof.

### FIELD OF THE INVENTION

The invention is generally directed to antimicrobial hydrogel dressings.

### BACKGROUND OF THE INVENTION

The National Institutes of Health reported that biofilms are present in 80% of known wound infections, and typically demonstrate increased resistance to antimicrobial, immunological, predatory, and chemical attack (Percival, S., et al., Wound Repair and Regeneration, 16(1):52-57 (2008); Percival, S. et al, Int Wound J., 9(5):488-93 (2012)). Biofilms are polymicrobial by definition, and studies have shown they can form in 10 hours or less, do not demonstrate typical local signs of acute infection, and resist many commercial topical agents and wound dressings. Inevitably, either mechanical or chemical debridement is required (Black, C. and Costerton, Surg. Clin. N. Am., 90(6):1147-1160 (2010)). Moreover, biofilm eradication often requires antibiotic solution concentrations many times higher than planktonic treatments (Ceti, H., et al., J Clin Microbiol., 37(6):1771-1776 (1999)).

Therefore, it is an object of the invention to provide antimicrobial wound dressings that are effective for treating biofilms in wounds.

It is another object of the invention to provide hydrogel wound dressings that are effective for treating biofilms in wounds.

### SUMMARY OF THE INVENTION

Wound dressings are provided that are antimicrobial and are effective for treating biofilms in wounds. One embodiment provides a multi-layer wound dressing including a silver releasing substrate, for example a substrate containing silver-coated fibers or yarns, optionally elastane, and a hydrogel layer containing a therapeutic substance or substances suitable for wound care such as: hyaluronic acid, hypochlorous acid, acrylic acid, ascorbic acid, algenic acid, boric acid, citric acid, acetic acid and derivatives or combinations thereof. The disclosed hydrogel containing dressings are capable of delivering a variety of therapeutic substances, including cleansers, surfactants, coagulants, growth factors, moisturizers, antimicrobials and the like to a wound site. The hydrogel layer optionally may contain a pattern and/or one or more apertures, and may be secured by an optional non-adherent netting. A preferred silver-releasing substrate contains 100% silver-coated nylon fibers or yarns. The silver-coated nylon can be a knitted, woven, compound, or complex fabric. Silver fibers also can be combined with non-silver fibers, elastane, or contained within non-woven fabrics. Any of these silver containing fabrics, optionally, can be non-adherent, may contain elastane and/or one or more apertures. In addition, the dressing optionally contains the following: an adhesive layer, a permeable or porous separation layer, a moisture regulation layer for absorbing or donating moisture, a film layer, and combinations thereof. The hydrogel of the disclosed dressings typically has a pH of about 2-7, and the dressings release 5-50 ppm of ionic silver into the wound or wound fluids within 24 hours.

One embodiment provides a wound contact dressing that has a silver-releasing conformable layer made of yarns or fibers containing multifilament nylon (FIGS. 1A and 1B). In one embodiment, at least a majority of the fibers or yarns are completely and circumferentially coated with metallic silver, for example by an electroless silver plating process. At least one side of the silver releasing conformable layer is at least partially coated with a hydrogel layer containing a therapeutic substance or substances such as 1-20% (w/v) acetic or citric acid or their derivatives or combinations thereof, and optionally a surfactant or surfactants such as benzethonium chloride, and a pH of approximately 2-7. Optionally, the dressing may also contain a permeable or porous separation layer between at least one hydrogel layer and the conformable layer. Optionally, the dressing may also contain a top film or separating layer over at least one hydrogel layer. The wound dressing releases approximately 5 -50 ppm of ionic silver within 24 hours into a wound or wound fluids when in contact with the wound. Depending upon the needs of the patient, either side of the dressing may contact the wound.

Another embodiment provides a wound contact dressing having two silver releasing conformable layers made of yams or fibers containing multiple filaments of nylon wherein at least a majority of fibers or yarns of nylon are coated with metallic silver, for example by an electroless plating process (FIG. 2A), and either conformable layer, optionally, may contain elastane. The wound dressing also contains a hydrogel layer largely on and/or between at least one of the conformable layers. The hydrogel layer contains, for example, a therapeutic substance or substances or derivatives or combinations thereof such as about 1-20% (w/v) citric acid, optionally a surfactant or surfactants and a pH of about 2-7. The wound dressing preferably releases about 5 to 50 ppm, more preferably about 10-35 ppm of ionic silver within 24 hours into a wound or wound fluids when the dressing is applied. The wound dressing is applied to the wound so that either conformable layer is in contact with the wound. In another embodiment, the dressing has a separation layer 2 in between each conformable silver releasing layer 1 and the hydrogel 3 (FIG. 2B). In another embodiment, the double contact dressing has a hydrogel on one outer surface as well as in between the conformable silver releasing layer (FIG. 2C). Another embodiment of the double contact dressing has a hydrogel on one outer surface with moisture regulation layer in between the two conformable layers (FIG. 2D).

Still another embodiment provides a wound dressing, namely an island dressing, that has a silver releasing conformable layer made of yarns or fibers containing multiple filaments of nylon wherein at least a majority of yarns or fibers of nylon are coated with metallic silver, for example by an electroless plating process (FIG. 3A), and the silver releasing conformable layer optionally may contain elastane. A hydrogel layer is placed on top of the silver releasing conformable layer and contains, for example, a therapeutic substance or substances such as about 1-20% (w/v) of citric or acetic acid or a derivative and/or combination thereof, optionally a surfactant or surfactants, and a pH of about 2-7. A moisture regulation layer is placed on top of the hydrogel. Optionally, a permeable or porous separation layer is placed between the hydrogel layer, and the moisture regulation layer. The moisture regulation layer can be a rayon or foam pad or the like. Optionally, a film layer is placed on top of the moisture regulation layer. An adhesive layer is on top of either the optional film layer or the moisture regulation layer and extends beyond the pad to adhere to healthy skin surrounding the wound. In this embodiment, the silver releasing conformable layer is in contact with the wound when the dressing is applied to the wound. The wound dressing preferably releases about 5 to 50 ppm, more preferably at least about 10 ppm of ionic silver within 24 hours into a wound or wound fluids when the wound dressing is applied.

Another embodiment provides a wound dressing, namely an island dressing, that has a silver releasing conformable layer made of yarns or fibers containing multiple filaments of nylon wherein at least a majority of fibers or yarns of nylon are coated with metallic silver, for example by an electroless plating process (FIG. 3B), and the silver releasing conformable layer optionally may contain elastane. The silver releasing conformable layer is sandwiched between a moisture regulation layer on one side, and a hydrogel layer on the other side. Optionally, a permeable or porous separation layer is placed between the silver releasing conformable layer and the moisture regulation layer. The hydrogel layer contains, for example, a therapeutic substance or substances such as about 1-20% (w/v) of ascorbic acid or derivatives and combinations thereof, optionally a surfactant or surfactants, and a pH of about 2-7. Optionally, a film layer is placed on top of the moisture regulation layer. The dressing has an adhesive layer on top of either the optional film or the moisture regulation layer that extends beyond the moisture regulation layer. The moisture regulation layer can be a rayon or foam pad or the like. In this embodiment, the dressing is applied to wound so that the hydrogel is in contact with the wound. The wound dressing preferably releases about 5 to 50 ppm, more preferably at least about 5 ppm of ionic silver within 24 hours into a wound or wound fluids when the wound dressing is applied.

Another embodiment provides a wound dressing, namely a pad dressing, that has a silver releasing conformable layer made of yarns or fibers containing multiple filaments of nylon wherein at least a majority of fibers or yarns of nylon are coated with metallic silver, for example by an electroless plating process (FIG. 4A), and the silver releasing conformable layer optionally may contain elastane. The dressing has a hydrogel layer on top of the silver releasing conformable layer and contains, for example, a therapeutic substance or substances such as about 1-25% (w/v) of hypochlorous acid and derivatives and/or combinations thereof, optionally a surfactant or surfactants, and a pH of about 2-7. A moisture regulation layer is on top of the hydrogel layer and can be a rayon or foam pad or the like. Optionally, a permeable or porous separation layer is placed between the hydrogel and the moisture regulation layer. Optionally, a layer of a film covers the moisture regulation layer. In this embodiment the dressing is applied to a wound so that the silver releasing conformable layer is in contact with the wound. The wound dressing preferably releases about 5 to 50, more preferably at least about 10 ppm of ionic silver within 24 hours into a wound or wound fluids when the wound dressing is applied.

Another embodiment provides a wound dressing, namely a pad dressing, that has a silver releasing conformable layer made of yarns or fibers containing multiple filaments of nylon wherein at least a majority of fibers or yarns of nylon are coated with metallic silver, for example by an electroless plating process (FIG. 4B), and the silver releasing conformable layer optionally may contain elastane. The dressing has a hydrogel layer beneath the silver releasing conformable layer and contains, for example a therapeutic substance or substances such as about 1-50% (w/v) of alginic acid or its derivatives and combinations thereof, optionally a surfactant or surfactants, and a pH of about 2-7. A moisture regulation layer is on top of the silver releasing conformable layer and can be a rayon or foam pad or the like. Optionally, a permeable or porous separation layer is placed between the silver releasing conformable layer and the moisture regulation layer. Optionally, a layer of a film covers the moisture regulation layer. Optionally, the hydrogel is secured with a netting. In this embodiment, the dressing is applied to a wound so that the hydrogel layer is in contact with the wound. The wound dressing preferably releases about 5 to 50 ppm, more preferably at least about 5 ppm of ionic silver within 24 hours into a wound or wound fluids when the wound dressing is applied.

### BRIEF DESCRIPTION OF THE DRAWINGS

FIG. 1A is a diagram of an exemplary hydrogel contact dressing. FIG. 1B shows another embodiment of a hydrogel contact dressing.

FIG. 2A shows another embodiment of a hydrogel double contact dressing. FIG. 2B shows another embodiment of dressing. In FIG. 2B, the dressing has a separation layer 2 in between each conformable silver releasing layer 1 and the hydrogel 3. FIG. 2C is another embodiment of the double contact dressing having a hydrogel on one outer surface as well as in between the conformable silver releasing layer. FIG. 2D shows another embodiment of the double contact dressing having a hydrogel on one outer surface with moisture regulation layer in between the two conformable layers.

FIG. 3A shows an exemplary hydrogel island wound dressing. FIG. 3B depicts another embodiment of a hydrogel island dressing.

FIG. 4A shows an exemplary hydrogel pad dressing. FIG. 4B shows another embodiment of the hydrogel pad dressing

FIGS. 5A and 5B are scanning electron micrographs of an exemplary silver-coated nylon substrate made of yams wherein the yams contain multiple longitudinal filaments. FIG. 5A is before and FIG. 5B is after 7 days of immersion in tryptic soy broth. The micrographs show the multiple longitudinal filaments in the yarns of the fabric.

### DETAILED DESCRIPTION OF THE INVENTION

### I Definitions

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

### II Antimicrobial Hydrogel Dressings

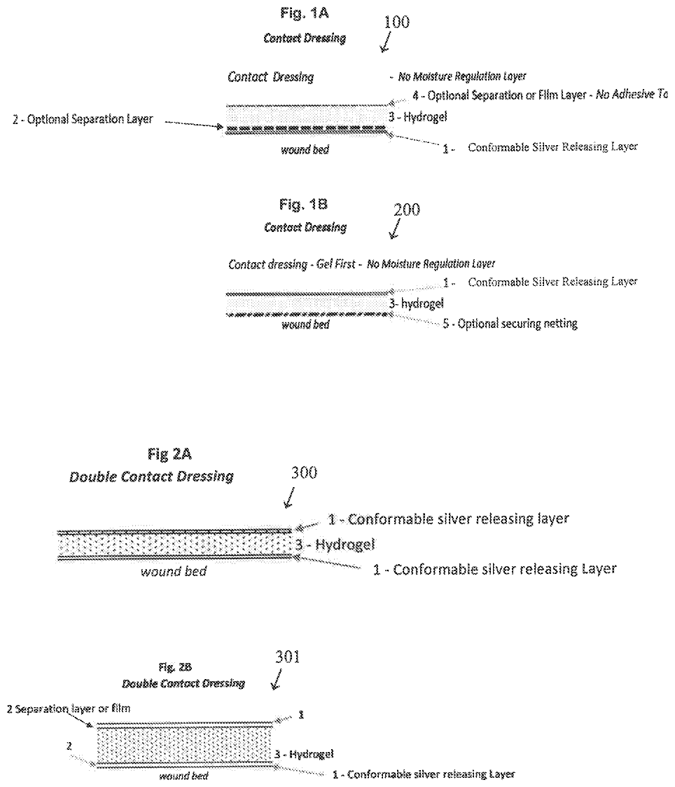

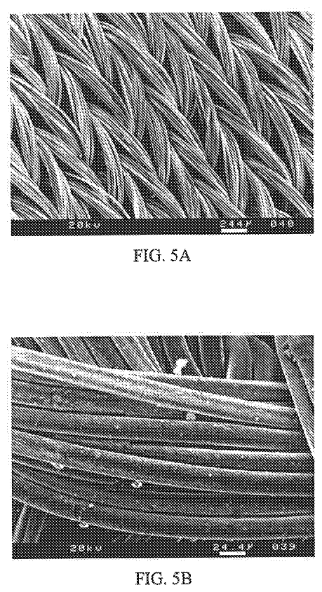

Several different embodiments of antimicrobial hydrogel dressings are provided. The thickness of the continuous or non-continuous hydrogel layer could be 2, 3, 4, 5, 6, 7, 8, 9, 10, 11, 12, 13, 14. 15, 16, 17, 18, 19, or 20 mm and optionally contain apertures of about 0.05 cm to 2 cm or be applied as a uniform or mixed pattern coating using shapes such as squares, circles or dots or like. The components of each embodiment are discussed individually below. FIG. 1A shows a diagram of an exemplary contact dressing 100. Dressing 100 has a silver releasing conformable bottom layer 1. In this and the following embodiments, the silver releasing conformable substrate can be substrate impregnated with, saturated with, or coated with metallic silver such that the silver releasing conformable substrate releases ionic silver when contact with a wound, wound fluid, or bodily fluid. In one embodiment the silver releasing conformable substrate a silver-coated substrate or silver coated fiber or silver metal fiber containing non-woven substrate. The silver fiber containing substrate 1 can be a conformable layer made of yarns or fibers containing multiple filaments of nylon wherein the multiple filaments of nylon are coated with metallic silver, for example by an electroless plating process. FIG. 5A shows an exemplary yarn made of multiple filaments wherein the multiple filaments are each individually and circumferentially coated with metallic silver and combine to form a yarn. The silver fiber containing substrate has a top side and a bottom side. Bottom side is in contact with the wound bed, and the top side is coated with hydrogel 3. The hydrogel 3 contains a therapeutic substance or substances or derivatives and/or combinations thereof such as 1- 20% (w/v) of citric acid or acetic acid or the like, optionally a surfactant or surfactants, and has a pH of about 2-7. In one embodiment, the silver fiber containing substrate 1 and the hydrogel 3 is separated by an optional separation layer 2. In still another embodiment, the hydrogel 3 has an optional separation or film layer 4 on top of the hydrogel 3. The dressing 100 preferably releases about 5 to 50, more preferably at least about 10 ppm of ionic silver within 24 hours into a wound or wound fluids when the wound dressing is applied.

FIG. 1B shows another embodiment of a hydrogel contact dressing 200. Dressing 200 is similar to dressing 100 except that in dressing 200 the silver releasing conformable substrate, for example a silver fiber-containing substrate 1 serves as the top layer. The middle layer is a hydrogel 3. The dressing optionally contains securing netting 5 separating the hydrogel 3 from the wound bed. Hydrogel 3 contains, for example, a therapeutic substance or substances such as 1- 20% (w/v) of citric acid or acetic acid, a surfactant or surfactants, and/or combinations or derivatives thereof and has a pH of about 2-7. The wound dressing 200 preferably releases about 5 to 50 ppm, more preferably at least about 5 ppm of ionic silver within 24 hours into a wound or wound fluids when the wound dressing is applied to a wound.

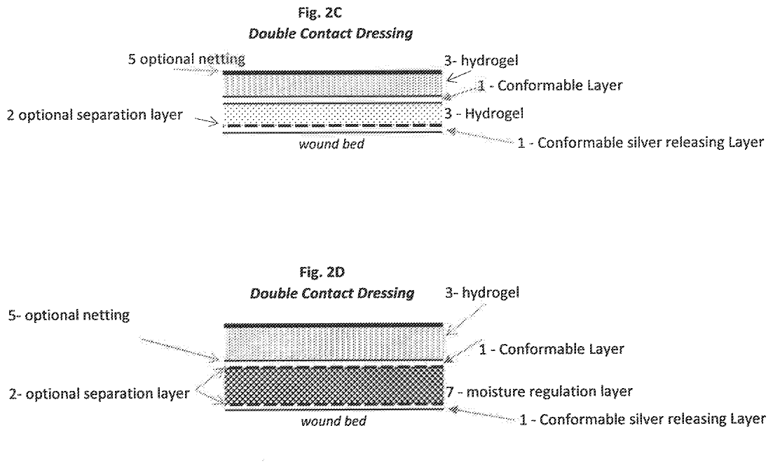

FIG. 2A shows another embodiment of a hydrogel double contact dressing 300. Dressing contains a silver releasing conformable top layer, for example a silver fiber containing conformable substrate 1 and a conformable bottom silver releasing layer, for example a silver fiber containing conformable substrate 1 separated by a hydrogel 3. The bottom silver releasing layer 1 is in contact with the wound bed. The hydrogel 3 contains, for example, a therapeutic substance or substances such as 1- 20% (w/v) of citric acid or acetic acid or derivatives and/or combinations or the like, optionally a surfactant or surfactants, and has a pH of about 2-7. The wound dressing 300 preferably releases about 5 to 50 ppm, more preferably at least about 5 ppm of ionic silver within 24 hours into a wound or wound fluids when the wound dressing is applied to a wound. FIG. 2B shows another embodiment 301of dressing. FIG. 2B shows a dressing 301 having a separation layer 2 separating each conformable layer 1 from the hydrogel 3. FIG. 2C is another embodiment 302 of a dressing having a hydrogel 3 on one outer surface with an optional secure netting 5 as well as in between two silver releasing, conformable layers 1. FIG. 2D is another embodiment 303 of a dressing having a hydrogel 3 on one outer surface with a moisture regulation layer 7 in between the two silver releasing conformable layers 1 as well two optional separation layers 2.

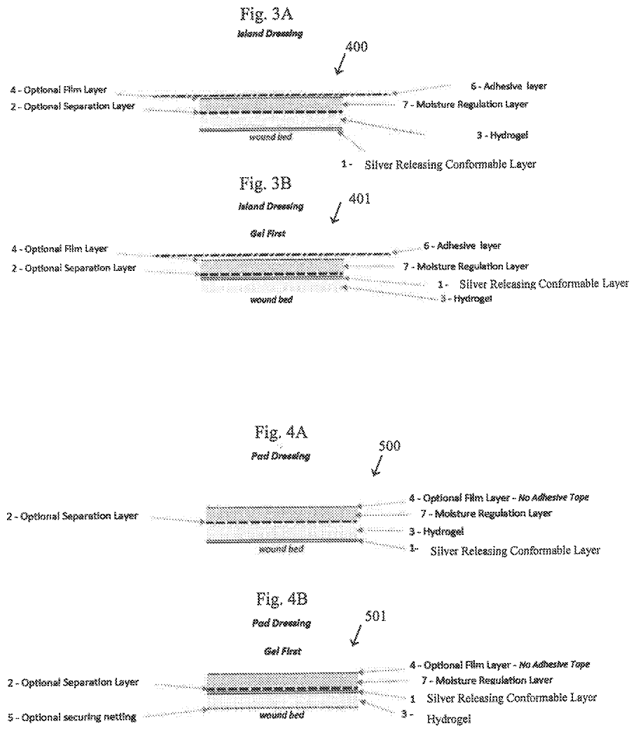

FIG. 3A shows an exemplary hydrogel island wound dressing 400. Dressing 400 has a silver releasing conformable bottom layer 1, for example a layer containing silver fibers, which is in contact with the wound bed. Hydrogel layer 3 is on top of bottom layer 1 and contains, for example, a therapeutic substance or substances such as 1- 20% (w/v) of citric acid or acetic acid, a surfactant or surfactants, and/or derivatives and/or combinations thereof or the like and has a of about 2-7. The dressing 400 optionally contains a separation layer 2 on top of hydrogel 3. A moisture regulation layer 7 is on top of optional separation layer 2 or hydrogel 3. Optional film layer 4 covers moisture regulation layer 7. An adhesive layer 6 covers optional film layer 4 or moisture regulation layer 7. The wound dressing 400 preferably releases about 5 to 50 ppm, more preferably at least about 10 ppm of ionic silver within 24 hours into a wound bed or wound fluids when the wound dressing is applied to a wound.

FIG. 3B depicts another embodiment 401 of a hydrogel island dressing similar to the dressing of FIG. 3A wherein hydrogel 3 is in contact with the wound and has conformable silver releasing substrate 1 on top of hydrogel 3. The dressing optionally has a separation layer 2 on top of silver releasing substrate, conformable layer 1. A moisture regulation layer 7 is on top of optional separation layer 2 or the silver releasing conformable layer 1. An optional film layer 4 is on top of moisture regulation layer 7. An adhesive layer 6 covers optional film layer 4 or moisture regulation layer 7. The hydrogel layer contains, for example, a therapeutic substance or substances such as 1- 20% (w/v) of citric acid or acetic acid, and/or a surfactant or surfactants and/or derivatives and/or combinations thereof or the like and has a pH of about 2-7. The wound dressing preferably releases about 5 to 50 ppm, more preferably at least about 5 ppm of ionic silver within 24 hours into a wound bed or wound fluids when the wound dressing is applied to a wound.

FIG. 4A shows an exemplary pad dressing 500 having a silver fiber containing silver releasing conformable layer 1, for example a layer containing silver coated fibers, as the layer that contacts the wound. Hydrogel 3 is on top of conformable layer 1. Optional separation layer 2 is on top of hydrogel 3. A moisture regulation layer 7 is on top of optional separation layer 2 or hydrogel 3. Optional film layer 4 covers moisture regulation layer 7. The hydrogel layer contains, for example, a therapeutic substance or substances such as 1- 20 (w/v) of citric acid or acetic acid or derivatives and/or combinations thereof or the like and has a pH of about 2-7. The wound dressing preferably releases about 5 to 50 ppm, more preferably at least about 10 ppm of ionic silver within 24 hours into a wound bed or wound fluids when the wound dressing is applied to a wound.

FIG. 4B shows another embodiment 501 of the pad dressing having hydrogel 3 as the layer that contacts the wound. Hydrogel 3 contains, for example, a therapeutic substance or substances such as 1- 20% (w/v) of citric acid or acetic acid or derivatives and/or combinations thereof or the like and has a pH of about 2-7. In one embodiment the dressing has an optional securing netting 5 that separates hydrogel 3 from the wound. Silver releasing conformable substrate 1, for example a layer that contains silver coated fibers, is on top of hydrogel 3. Optional separation layer 2 is on top of silver releasing conformable substrate 1. Moisture regulation layer 7 is on top of optional separation layer 2 or silver releasing conformable layer 1. Optional film layer 4 is on top of moisture regulation layer 7. The wound dressing 500 preferably releases about 5 to 50, more preferably at least about 5 ppm of ionic silver within 24 hours into a wound bed when the wound dressing is applied.

A. Hydrogel

Hydrogel 3 can be a three dimensional network of hydrophilic polymers.

In one embodiment, hydrogel 3 is a thin gel pattern or web of low viscosity or one that changes and absorbs, degrades, deforms, dissolves, hydrolyzes, or the like in response to contact with wound fluids, wound tissue, or wound pH.

Types of hydrogel dressings include amorphous or free flowing hydrogels that are typically which can be saturated into a gauze pad, sponge of fabric. Lastly, there are sheet hydrogels which are a combination of gel held together by a thin fiber mesh. One example is a hydrogel dressing made of polyurethane polymers containing about 60% water and can absorb excess wound exudate and locks it into the gel structure.

Preferred hydrogels 3 conform to the body shape, do not adhere to the wound bed, are permeable to gas and water, contain, for example, a therapeutic substance or substances such as 1 to 20% (w/v) of acetic or citric acid or the like, derivatives and/or combinations thereof, optionally a surfactant or surfactants, and have a pH of about 2-7, or have a combination of these features.

The disclosed hydrogel dressings contain one or more hydrogel layers. The hydrogel includes one or more gelling agents. Exemplary gelling agents that can be used in the disclosed dressing include, but are not limited to acacia, alginic acid, bentonite, Carbopols® (now known as carbomers), carboxymethylcellulose, ethylcellulose, gelatin, hydroxyethylcellulose, hydroxypropyl cellulose, magnesium aluminum silicate (Veegum®), methylcellulose, poloxamers (Pluronics®), polyvinyl alcohol, sodium alginate, ragacanth, and xanthan gum. Though each gelling agent has some unique properties, there are some generalizations that can be made.

Some gelling agents are more soluble in cold water than in hot water. Methylcellulose and poloxamers have better solubility in cold water while bentonite, gelatin, and sodium carboxymethylcellulose are more soluble in hot water. Carbomers, tragacanth, and alginic acid gels are made with tepid water.

Some gelling agents (carbomers) require a “neutralizer” or a pH adjusting chemical to create the gel after the gelling agent has been wetted in the dispersing medium.

1. Carbomer

Carbomers can also be used in the disclosed dressings, and carbomer is a generic name for a family of polymers known as Carbopol®. Carbopols® that were first used in the mid-1950s. As a group, they are dry powders with high bulk densities, and form acidic aqueous solutions (pH around 3.0). They thicken at higher pHs (around 5 or 6). They will also swell in aqueous solution of that pH as much as 1000 times their original volume. Their solutions range in viscosity from 0 to 80,000 centipoise (cps). Some examples of this group of gelling agents are:

Carbopol® 910 has viscosity of 3,000-7,000 cps and is effective in low concentrations and provides a low viscosity formulation;

Carbopol® 934 has a viscosity of 30,500-39,400 cps and is effective in thick formulations such as emulsions, suspensions, sustained-release formulations, transdermals, and topicals;

Carbopol® 934P has a viscosity of 29,400-39,400 cps with the same properties as 934, and is typically used in pharmaceutical formulations;

Carbopol® 940 has a viscosity of 40,000-60,000 cps and is effective in thick formulations, has very good clarity in water or hydroalcoholic topical gels; and

Carbopol® 941 has a viscosity of 4,000-11,000 cps and produces low viscosity gels with very good clarity.

Carbomer polymers are best introduced into water by slowly sprinkling a sieved powder into the vortex created by rapid stirring. This should prevent clumping. Once all of the powder has been added, the stirring speed should be reduced to decrease the possibility of entrapping air bubbles in the formulation.

When the carbomer is dispersed, the solution will have a low pH. A “neutralizer” is added to increase the pH and cause the dispersion to thicken and gel. Some neutralizing agents are sodium hydroxide, potassium hydroxide, and triethanolamine. If the inorganic bases are used to neutralize the solution, a stable water soluble gel is formed. If triethanolamine is used, the gel can tolerate high alcohol concentrations. The viscosity of the gel can be further manipulated by propylene glycol and glycerin (to increase viscosity) or by adding electrolytes (to decrease viscosity).

2, Cellulose Derivatives

The cellulose derivatives (methylcellulose, hydroxyl-ethylcellulose, hydroxypropylcellulose, hydroxypropyl-methylcellulose, and carboxymethylcellulose) can also be used in the disclosed dressings. There are some commonalties in these compounds, and each one has their unique properties.

Methylcellulose has a viscosity of 1500 cps and makes thinner gels with high tolerance for added drugs and salts. It is compatible with water, alcohol (70%), and propylene glycol (50%) and hydrates and swells in hot water. The powder is dispersed with high shear in about ⅓ of the required amount of water at 80° C. to c90° C. Once it is dispersed, the rest of the water (as cold water or ice water) is added with moderate stirring. Maximum clarity, hydration, and viscosity will be obtained if the gel is cooled to 0-10° C. for about an hour.

Hydroxyethylcellulose makes thinner gels that are compatible with water and alcohol (30%). It hydrates and swells in cool water (about 8-12 hours). It forms an occlusive dressing when lightly applied to the skin and allowed to dry

Hydroxypropylcellulose makes thinner gels with high tolerance for added drugs and salts and is compatible with alcohols and glycols. It hydrates and swells in water or hydroalcoholic solution. The powder is sprinkled in portions into water or hydroalcoholic solution without stirring and allowed to thoroughly wet. After all of the powder is added and hydrated (about 8 - 12 hours), the formulation can be stirred or shaken. It is a good gelling agent if 15% or more of an organic solvent is needed to dissolve the active drug.

Hydroxypropylmethylcellulose makes thicker gels but has a lower tolerance for positively charged ions. It is compatible with water, alcohol (80%) and disperses in cool water. It is a good gelling agent for time released formulations.

Carboxymethylcellulose is generally used as the sodium salt. It makes thicker gels but has less tolerance than hydroxypropylmethylcellulose. It has a maximum stability at pH 7-9 and is compatible with water and alcohol. It disperses in cold water to hydrate and swells. It is then heated to about 60° C. Maximum gelling occurs in 1-2 hours.

Poloxamer (Pluronics®) are copolymers of polyoxyethylene and polyoxypropylene. They will form thermoreversible gels in concentration ranging from 15% to 50%. This means they are liquids at cool (refrigerator) temperature, but are gets at room or body temperature. Poloxamer copolymers are white, waxy granules that form clear liquids when dispersed in cold water or cooled to 0-10° C. overnight.

3, Ionic Hydrogels

Ionic polysaccharides, such as alginates or chitosan, can be used in the disclosed dressings. In one embodiment, the hydrogel is produced by cross-linking the anionic salt of alginic acid, a carbohydrate polymer isolated from seaweed, with ions, such as calcium cations. The strength of the hydrogel increases with either increasing concentrations of calcium ions or alginate. For example, U.S. Pat. No. 4,352,883 describes the ionic cross-linking of alginate with divalent cations, in water, at room temperature, to form a hydrogel matrix,

In general, these polymers are at least partially soluble in aqueous solutions, e.g., water, or aqueous alcohol solutions that have charged side groups, or a monovalent ionic salt thereof. There are many examples of polymers with acidic side groups that can be reacted with cations, e.g., poly (phosphazenes), poly(acrylic acids), and poly(methacrylic acids). Examples of acidic groups include carboxylic acid groups, sulfonic acid groups, and halogenated (preferably fluorinated) alcohol groups. Examples of polymers with basic side groups that can react with anions are poly(vinyl amines), poly(vinyl pyridine), and poly(vinyl imidazole).

Polyphosphazenes can also be used in the disclosed dressings and are polymers with backbones consisting of nitrogen and phosphorus atoms separated by alternating single and double bonds. Each phosphorus atom is covalently bonded to two side chains. Polyphosphazenes that can be used have a majority of side chains that are acidic and capable of forming salt bridges with di- or trivalent cations. Examples of acidic side chains are carboxylic acid groups and sulfonic acid groups.

Polyphosphazenes that erode in vivo have at least two different types of side chains: acidic side groups capable of forming salt bridges with multivalent cations, and side groups that hydrolyze under in vivo conditions, e.g., imidazole groups, amino acid esters, glycerol, and glucosyl. Degradable polymers, i.e., polymers that dissolve or degrade within a period that is acceptable in the desired application (usually in vivo therapy), will degrade in less than about five years and most preferably in less than about one year, once exposed to a physiological solution of pH 6-8 having a temperature of between about 25° C. and 38° C. Hydrolysis of the side chain results in erosion of the polymer. Examples of hydrolyzing side chains are unsubstituted and substituted imidizoles and amino acid esters in which the side chain is bonded to the phosphorous atom through an amino linkage.

Methods for synthesis and the analysis of various types of polyphosphazenes are described in U.S. Pat. Nos. 4,440,921, 4,495,174, and 4,880,622. Methods for the synthesis of the other polymers described herein are known to those of ordinary skill in the art. See, for example Concise Encyclopedia of Polymer Science and Engineering, J. I. Kroschwitz, editor (John Wiley and Sons, New York, N.Y., 1990). Many polymers, such as poly(acrylic acid), alginates, and PLURONICS™, are commercially available. Water soluble polymers with charged side groups are cross-linked by reacting the polymer with an aqueous solution containing multivalent ions of the opposite charge, either multivalent cations if the polymer has acidic side groups, or multivalent anions if the polymer has basic side groups. Cations for cross-linking the polymers with acidic side groups to form a hydrogel include divalent and trivalent cations such as copper, calcium, aluminum, magnesium, and strontium. Aqueous solutions of the salts of these cations are added to the polymers to form soft, highly swollen hydrogels.

Anions for cross-linking the polymers to form a hydrogel include divalent and trivalent anions such as low molecular weight dicarboxylate ions, terepthalate ions, sulfate ions, and carbonate ions. Aqueous solutions of the salts of these anions are added to the polymers to form soft, highly swollen hydrogels, as described with respect to cations.

4, Temperature-Dependent Hydrogels

Temperature-dependent, or thermosensitive, hydrogels can be used in the disclosed dressings. These hydrogels have so-called “reverse gelation” properties, i.e., they are liquids at or below room temperature, and gel when warmed to higher temperatures, e.g., body temperature. Thus, these hydrogels can be easily applied at or below room temperature as a liquid and automatically form a semi-solid gel when warmed to body temperature. Examples of such temperature-dependent hydrogels are PLURONICS® (BASF-Wyandotte), such as polyoxyethylene-polyoxypropylene F-108, F-68, and F-127, poly (N-isopropylacrylacrylamide), and N-isopropylacrylamide copolymers.

These copolymers can be manipulated by standard techniques to alter physical properties such as their porosity, rate of degradation, transition temperature, and degree of rigidity. For example, the addition of low molecular weight saccharides in the presence and absence of salts affects the lower critical solution temperature (LCST) of typical thermosensitive polymers. In addition, when these gels are prepared at concentrations ranging between 5 and 25% (W/V) by dispersion at 4° C., the viscosity and the gel-sol transition temperature are affected, the gel-sol transition temperature being inversely related to the concentration. These gels have diffusion characteristics capable of allowing cells to survive and be nourished.

U.S. Pat. No. 4,188,373 describes the use of PLURONIC™ polyols in aqueous compositions to provide thermal gelling aqueous systems. U.S. Pat. Nos. 4,474,751, '752, '753, and 4,478,822 describe drug delivery systems that utilize thermosetting polyoxyalkylene gels. With these systems, both the gel transition temperature and/or the rigidity of the gel can be modified by adjusting the pH and/or the ionic strength, as well as by the concentration of the polymer.

5. pH-Dependent Hydrogels

Other hydrogels suitable for use with the disclosed dressings are pH-dependent. These hydrogels are liquids at, below, or above specific pH values, and gel when exposed to specific p1-I values, e.g., 7.35 to 7.45, which is the normal pH range of extracellular fluids within the human body. Thus, these hydrogels can be easily administered as a liquid and automatically form a semisolid gel when exposed to body pH. Examples of such pH-dependent hydrogels are TETRONICS™ (BASF-Wyandotte) polyoxyethylene-polyoxypropylene polymers of ethylene diamine, poly(diethyl aminoethyl methacrylate-g-ethylene glycol), and poly(2-hydroxymethyl methacrylate) These copolymers can be manipulated by standard techniques to affect their physical properties.

6, Light Solidified Hydrogels

Other hydrogels that can be used in the disclosed dressings are solidified by either visible or ultraviolet light. These hydrogels are made of macromers including a water soluble region, a biodegradable region, and at least two polymerizable regions as described in U.S. Pat. No, 5,410,016. For example, the hydrogel can begin with a biodegradable, polymerizable macromer including a core, an extension on each end of the core, and an end cap on each extension. The core is a hydrophilic polymer, the extensions are biodegradable polymers, and the end caps are oligomers capable of cross-linking the macromers upon exposure to visible or ultraviolet light, e.g., long wavelength ultraviolet light.

Examples of such light solidified hydrogels include polyethylene oxide block copolymers, polyethylene glycol polylactic acid copolymers with acrylate end groups, and 10K polyethylene glycol-glycolide copolymer capped by an acrylate at both ends. As with the PLURONIC™ hydrogels, the copolymers comprising these hydrogels can be manipulated by standard techniques to modify their physical properties such as rate of degradation, differences in crystallinity, and degree of rigidity. Light solidified hydrogels are useful, for example, for direct painting of the hydrogel-cell mixture onto damaged tissue.

7 Biofilm Degradation Agents

The disclosed hydrogel dressings include a biofilm degradation agent or agents in the hydrogel 3. Exemplary biodegradation agents include, but are not limited to EDTA, acetic acid, citric acid, surfactants such as benzethonium chloride and combinations thereof.

EDTA is ethylene-diaminetetraacetic acid is a chelating agent that binds metals including but not limited to calcium ions, magnesium ions, and iron ions.

Acetic acid is a carboxylic acid having the following formula CH₃COOH.

Citric acid is an acid found in citrus fruits. Its molecular formula is CH₂COOH—C(OH)COOH—CH₂COOH. Salts of citric acid chelate calcium.

In one embodiment, the biofilm degradation agent is present in the hydrogel in about 0.1 to 20 percent (w/v), preferably 1 to 6% (w/v). In another embodiment, the biofilm degradation agent is present in about 5, 10, 15, 20%

In one embodiment, the disclosed dressing includes a therapeutically effective amount of benzethonium chloride. Benzethonium chloride has surfactant, antiseptic, and anti-infective properties, and it is used as a topical antimicrobial agent in first aid antiseptics. It's IUPAC name is benzyl-dimethyl-[2-[2-[4-(2.4,4-trimethylpentan-2-yl)phenoxy[ethoxy]ethyl]azanium; chloride.

### B. Silver-coated Substrates

1, Polyamides

In some embodiments, the silver releasing conformable substrate 1 is preferably a flexible and conformable substrate made of silver-coated polyamide. Substrate 1 can contain silver coated fibers and filaments. A preferred polyamide is nylon. The term “nylon” refers to a family of linear polyamides. The family of nylons includes several different types. Nylon 6/6, nylon 6, nylon 6/10, nylon 6/12, nylon 11, nylon 12, and nylon 6-6/6 copolymer are the most common. Of these, nylon 6/6 and nylon 6 are the most commonly used. The numbers refer to how many methyl units (—CH₂—) occur on each side of the nitrogen atoms (amide groups). The difference in number of methyl units influences the property profiles of the various nylons. The properties of some nylons are provided in Table 1 below.

TABLE 1

| | A.S.T.M | NYLON | NYLON | NYLON | NYLON |
|---|---|---|---|---|---|
| | Test | TYPE | TYPE | TYPE | CAST |
| PROPERTIES | Method | 6 | 6/6 | 6/12 | TYPE 6 |
| Specific Gravity | D792 | 1.12-1.14 | 1.14-1.1 | 1.06 | 1.15 |
| Water Absorption | D570 | 2.9 | 1.24 | 0.25 | [symbol] |
| Method A | | | | | |
| Tensile strength at | D638 | 9.4 | 12 | 8.8 | 11-14 |
| yield, 1000 psi | | | | | |
| Elongation at yield, % | D638 | 25 | >150 | 7 | 10 |
| Elastic Modulus in | D638 | [symbol] | 4.4 | [symbol] | 3.5-4.5 |
| Tension, 10~5 psi | | | | | |
| Flexural Strength at | D790 | NO | 16 | NO | 16-17.5 |
| yield, 1000 psi | | YIELD | | YIELD | |
| Elastic modulus in | D790 | 1.50 | 4.1 | 2.95 | [symbol] |
| flexure, 10~5 psi | | | | | |
| Rockwell Hardness | D785 | R104 | 88 | R114 | R112 |
| (Method A) | | | | | |
| Izod impact | D256 | 2.2 | 1.2 | 1.5 | [symbol] |
| strength, ft-lb/in. | | | | | |
| notch ⅛ in. | | | | | |
| specimen | | | | | |
| Deform. under | D621 | [symbol] | 0.8 | 1.6 | 0.5-1.0 |
| load (2000 psi; | | | | | |
| 122 f), % | | | | | |
| Deflection | D648 | 340 | 450 | 356 | 400 |
| temperature, F. at 66 psi | | | | | |
| fiber stress | | | | | |
| Max recommended | [symbol] | 175 | 270 | 290 | 200-225 |
| service Temp., F. | | | | | |
| continuous use | | | | | |
| Coeff. of Linear | D696 | 4 × 10~5 | 4.5 × 10~5 | 5 × 10~5 | 5.0 × 10~5 |
| Thermal Expansion, F. | | | | | |
| Dielectric strength, | D149 | [symbol] | 555 | 650 | 500 |
| v/mil, short time | | | | | |
| Dielectric constant | D150 | 7.2 | 4.0 | 4.0 | 3.7 |
| at 60 Hertz | | | | | |
| Dielectric constant | D150 | 3.7 | 3.5 | 3.5 | 3.7 |
| at 1 Mega Hertz | | | | | |
| Dissipation factor, | D150 | [symbol] | 0.02 | .02 | [symbol] |
| at 60 Hertz | | | | | |
| Dissipation factor, | D150 | 0.12 | 0.03 | 0.2 | [symbol] |
| at 1 Mega Hertz | | | | | |
| Volume resistivity, | D257 | 10~12 | 10~15 | 10~15 | [symbol] |
| ohm-cm | | | | | |
| Arc resistance (SS | D495 | [symbol] | 123 | [symbol] | [symbol] |
| Electrode), sec. | | | | | |

The silver-coated substrate or silver containing non-woven substrate can comprise yarns or fibers of nylon. Each yarn or fiber of nylon includes multiple fibers or filaments of nylon. FIGS. 5A and 5B show an exemplary silver-coated substrate entirely made of yarns of nylon wherein the yarns are made of multiple longitudinal filaments. In one embodiment, each longitudinal filament is individually and uniformly coated with metallic silver by an electroless plating process. By using multiple longitudinal filaments to form yarns, the amount of surface area coated with metallic silver is significantly increased and allows for therapeutically effective amounts of silver ions to be released from the substrate and into the wound.

In one embodiment, the hydrogel dressing passively releases 5 to 50 ppm of ionic silver into a wound or wound fluids within 24 hours. Another embodiment of the hydrogel dressing releases 10 to 35 ppm of ionic silver into a wound or wound fluids. Still another embodiment of the hydrogel dressing releases 15 ppm of ionic silver into the wound or wound fluids. Passive release of silver ions from the dressing means that no electric current is applied to the dressing to force silver ions into the wound or wound fluids. In another embodiment the wound dressing releases about 5, 10,15, 20, 25, 30, 35, 40, 45, or 50 ppm of ionic silver in to the wound or wound fluids.

In one embodiment, the disclosed wound dressings release an effective amount of ionic silver into a wound or wound fluids to reduce the amount of microorganisms in the wound to less than 10⁵ CFU/ml within about 72 hours. In another embodiment, the disclosed wound dressings release an effective amount of ionic silver into a wound or wound fluids to reduce the amount of microorganisms in the wound to less than 10⁵ CFU/ml for at least 12 hours, 1 day, 2 days, 3 days, 4 days, 5 days, 6 days, or 7 days.

In one embodiment, the silver fiber containing substrate may contain elastane filaments, fibers, or yarns as well as apertures

The release of silver ions from the disclosed hydrogel dressings can be determined using one or more of the following tests. Test 1 begins by placing a 5×5 cm sample of the hydrogel dressing in 20 mL of simulated wound fluid (SWF) (2% bovine albumin, 0.02 M calcium chloride dihydrate, 0.4 M sodium chloride, 0.08 M tris-methylamine in de-ionized water, p1-I 7.5) and incubated in a circulating water bath at 37° C., 60 rpm for 7 days. Spent SWF is replaced every 24 hours with an equal volume of fresh SWF: the spent solution was retained for atomic absorption spectrometry analysis (Perkin Elmer Analyst 200).

Test 2 begins with a two piece set of approximately 3 inch plastic hoops (7.5cm), approximately 4 cm diameters of circular dressing is draped over the inner hoop and secured by placing the inner hoop with dressing within the clamp containing outer hoop and securely pinching the dressing between the two hoops. The dressing containing conjoined hoops is then submerged into a covered, stirrer containing Teflon beaker or equivalent.

Atomic Absorption Spectrophotometry is used to determine the metal concentration within a solution of Tryptic Soy Broth at 37° C. Given the low levels of silver likely to be released from these materials, flasks and sample containers that do not adsorb silver ions on their surfaces should be used in the analysis. In addition, sterility of the test media over the course of the testing must be ensured to eliminate erroneous low silver readings.

Instrumental Conditions:

Perkin-Elmer Model 100 Aanalyst AA, wavelength 328.1 nm, 0.7 nm slits width, 3 sec sample time, 2 replicate measurements. The air-acetylene flame should be rich, blue, oxidizing,

Materials:
1. Tryptic Soy Broth
2. HPLC grade water (distilled/deionized, >18 M-ohms/cm)
3. Nitric Acid (UltraPure, ACS Reagent Grade)
4. 1000 ppm Ag+ standard solution
5. 0.2 um Teflon Whatman UniPrep Syringeless filters (Fisher Scientific #090923-5)
6. Whatman Bugstopper Vents Caps (Whatman #67136010, Fisher Scientific #09-830-32)
7. Teflon 250 mL Erlenmeyer Flasks (Nalgene #4106 0250, Fisher Scientific #10-040-16C)
8. Teflon 10 mL Beakers (Fisher Scientific #02-586-1D)
9. Teflon FEP Bottles, 125 mL (Nalgene #2100 0004, Fisher Scientific π02-924-15A)

### Step-by-Step Procedure:

1. Prepare 100 mL of 4.0, 12.0, and 24.0 ppm Ag+ standard solutions by diluting 0.40 mL, 1.2 mL, or 2.4 mL portions, respectively, of 1000 ppm Ag+ solution to the mark in a 100 Ml volumetric flask with 0.10% nitric acid solution. Transfer these solutions to dark, tightly capped Teflon bottles for long-term storage/use.
2. Dissolve 30 grams of Tryptic Soy Broth (TSB) in boiled HPLC grade water to make 1.0 L solution. Cover tightly and allow to cool.
3. Sterilize by autoclaving, chemical treating, or other method, Teflon

Erlenmeyer flasks to be used in the analysis. Transfer exactly 250 mL of TSB to each Teflon Erlenmeyer flask. Immediately stopper the flasks with Whatman Bugstopper™ filter vents and place the flasks in a thermostated water bath or suitable environmental chamber set to 37° C. The flasks should be stirred or agitated at 150 rpm/min. Allow adequate time for the temperature of the solutions to equilibrate.
4. Wearing sterile gloves, obtain exactly 50 cm2 of each test fabric. Quickly uncap a flask and insert the test sample into the TSB solution. Recap immediately and start the timer. Be sure that the fabric does not impede a stirrer.
5. At 1, 2, 4, 8. 16. 24, 48, 72, 96, 120, and 144 hour intervals, stir each TSB solution with a stirring rod for 30 seconds and remove a 1.5 mL aliquot for silver analysis by AA. Be sure to use a clean stirring rod and pipet for each solution so the samples do not become cross-contaminated. The aliquots should be placed in a small Teflon beaker and analyzed immediately. If any visible particles of silver or fiber, or any bacterial growth is apparent in the TSB solutions, the samples must be filtered with 0.2 um filters before further analysis. Prepare the AA for use with HPLC water as a blank and the 4, 12, and 24 ppm standards prepared previously. Record Ag results of the unknown samples to the nearest 0.1 ppm along with RSD result.
Note: Nitric acid is not added to the 1.5 mL test solution aliquoits in this procedure since the AA is to take place immediately. There is significant concern that the addition of nitric acid may dissolve particulate silver that has fallen off the test dressings, giving rise to erroneously high TSB solution silver levels. By following this procedure, with filtering of the solutions, only ionic silver levels will be measured.

2. Electroless Plating

Electroless plating, also known as chemical or auto-catalytic plating, is a non-galvanic plating method that involves several simultaneous reactions in an aqueous solution, which occur without the use of external electrical power. (Schlesinger, M. (2010). Electroless and Electrodeposition of Silver. In Mordechay et al. (Ed.), Modern Electroplating, (5^{th} Ed.) John Wiley & Sons, Inc., Chapter 5.). Once the substrate, preferably plastic such as nylon, to be coated is prepared, it is immersed in an electroless bath. The electroless bath deposits a thin, adherent metallic silver film on the plastic surface by chemical reduction by using a semi-stable solution containing a silver salt such as silver nitrate, a reducer, a complex or for the silver, a stabilizer and a buffer system. (See Kuzmik, J. (1990) Plating on Plastics. In Mallory, G. et al., (Ed.) Electroless Plating: Fundamentals and Applications, American Electroplaters and Surface Finishers Society, Chapter 14.)

Silver coating on each filament is about 0.1 to about 5.0 mm in thickness. In a preferred embodiment, the silver metal coating is about 0.75 to about 1-2 mm.

The content of silver in the disclosed wound dressings can be about 500 to 5550 mg/100 cm². The total extractable silver content of the wound dressing can be determined following acid digestion of the sample using a technique called inductively coupled plasma optical emission spectroscopy (ICP-OES) or reasonably approximated by the difference in substrate weight per 100 cm² before and after electroless plating.

3. Apertures

Apertures can be present in the silver releasing conformable substrate, for example a silver-coated substrate or silver coated fiber containing substrate 1 and allow from 1 to 200 cc /24 hs/100 cm² of fluids or exudate to pass via capillary action or negative pressure therapy through the aperture in the silver-coated or silver fiber containing substrate 1.

The apertures can be of any geometric shape including, but not limited to circular, square, diamond, or star shaped. The apertures can have a length, width, diameter or axis of about 0.05 cm to about 2 cm.

C. Moisture Regulation Layer

Some embodiments of the disclosed hydrogel dressing include a moisture regulation layer 7. Moisture regulation can be used for absorbing or providing moisture to the wound dressing or wound. Exemplary moisture regulation layers can be rayon or foam pads or the like. Exemplary foam pads are made from polyurethane. The moisture regulation layer optionally contains apertures, is optionally conformable, and may contain silver coated fibers. In one embodiment the moisture regulation layer is conformable.

The moisture regulation layer maybe include a foam, a sponge or sponge-like material, cellulosic materials, cotton, rayon, polyvinyl alcohol, polyvinyl acetate, polyethylene oxide, polyvinyl pyrrolidone, polyurethane hydrocolloids, alginates, hydrogels, hydrocolloids, hydrofibrils, collagens or any combinations thereof.

D. Adhesive layers

Certain embodiments of the disclosed wound dressing have an adhesive layer 6. Adhesive layer 6 can contain variety of glues, adhesives, bonding agents, or cements. For example, the disclosed island hydrogel dressings can be attached to the wound using cyanoacrylate based adhesives such as methyl 2-cyanoacrylate, ethyl-2-cyanoacrylate, n-butyl cyanoacrylate, 2-octyl cyanoacrylate, or the like. Similarly, medical adhesives, skin glues, biological glues, and related products may be used to attach the wound dressing to the wound. In some cases, a gelatin solution or a collagen solution can be used. A preferred adhesive is acrylic adhesive. Other suitable adhesives include silicone, polyurethane, or hydrocolloid adhesives.

E. Thin Films

The thin films 4 can be a polymer film for example polyurethane film. Other suitable polymers include, but are not limited to neoprene, nylon, polyvinyl chloride (PVC or vinyl), polystyrene, polyethylene, polypropylene, polyacrylonitrile, PVB, and silicone. The films are permeable, porous or occlusive. In one embodiment, the films function as a physical barrier or vapor barrier. In another embodiment the films function as a gateway to add liquids.

F. Securing Netting

The securing netting can be a polymer such as nylon or polyethylene. The netting or mesh helps secure the hydrogel to the silver-fiber substrate 1. The netting or mesh is of medical grade and helps prevent the dressing from adhering to the wound. Medical netting is commercially available.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

1. A wound dressing comprising:
at least one conformable layer comprising:
yarns or fibers containing multiple filaments of nylon, wherein at least the majority of yams or fibers are completely and circumferentially coated with metallic silver by an electroless silver plating process, where at least one side of the conformable layer is at least partially coated with hydrogel or adjoined by a hydrogel layer comprising a therapeutic substance or substances or their derivatives, optionally a surfactant or surfactants, and has a pH of approximately 2-7, inhibits or reduces biofilm formation, and
wherein the wound dressing releases at least about 5 ppm of ionic silver within 24 hours into a wound or wound fluids when in contact with the wound or wound fluids.

2. The wound dressing of claim 1, wherein the therapeutic substance or substances comprises hyaluronic acid, hypochlorous acid, ascorbic acid, acrylic acid, algenic acid, boric acid, citric acid, or acetic acid, cleansers, coagulants, growth factors, surfactants, moisturizers, antimicrobials, or derivatives and/or combinations thereof

3. The wound dressing of claim 1, wherein the hydrogel has a thickness of about 2-20 mm, and optionally may be secured by open netting or film.

4. The wound dressing of claim 1, further comprising a moisture regulation layer for absorbing or donating moisture, and optionally contains silver-coated fibers.

5. The wound dressing of claim 1, optionally containing a film top layer above the moisture regulation layer or hydrogel.

6. The wound dressing of claim 1, optionally containing one or more permeable or porous separation layer(s) between any or all dressing layers.

7. The wound dressing of claim 1, wherein the moisture regulation layer comprises a foam, a sponge or sponge-like material, cellulosic materials, cotton, rayon, polyvinyl alcohol, polyvinyl acetate, polyethylene oxide, polyvinyl pyrrolidone, polyurethane hydrocolloids, alginates, hydrogels, hydrocolloids, hydrofibrils, collagens or any combinations thereof.

8. The wound dressing of claim 1, wherein the conformable layer contains elastane.

9. The wound dressing of claim 1, wherein the dressing comprises-two layers of conformable substrate separated by a hydrogel, and,
wherein dressing optionally contains separation layers between any and all layers.

10. An island dressing, comprising:
a conformable layer comprising:
yams or fibers containing multiple filaments of nylon, wherein at least a majority of the yarns or fibers are completely and circumferentially coated with metallic silver by an electroless silver plating process, and
wherein the conformable layer optionally comprises elastane, and &
wherein at least one side of the conformable layer is at least partially coated with hydrogel comprising a therapeutic substance of claim 2 and has a pH of approximately 2-7, and inhibits or reduces biofilm formation, and
a moisture regulation layer on top of either the comfortable layer or hydrogel layer, and
an adhesive layer covering the moisture regulation layer,
wherein the island dressing releases at least about 5 ppm of ionic silver within 24 hours into a wound or wound fluids when in contact with the wound or wound fluids.

11. The island dressing of claim 10, further comprising one or more separation layers separating the hydrogel from either the conformable layer and/or the moisture regulation layer.

12. The island dressing of claim 10, further comprising a film layer between the adhesive and moisture regulation layer.

13. A pad dressing comprising:
a conformable layer comprising:
yams or fibers containing multiple filaments of nylon, wherein at least a majority of the yarns or fibers are completely and circumferentially coated with metallic silver by an electroless silver plating process,
a hydrogel on top of or below the conformable layer, wherein the hydrogel comprises a therapeutic substance of claim 2 and has a pH of approximately 2-7, inhibits or reduces biofilm formation, and a moisture regulation layer on top of the hydrogel or the conformable layer,
wherein the pad dressing releases at least about 5 ppm of ionic silver within 24 hours into a wound or wound fluids when in contact with the wound or wound fluids.

14. A pad dressing of claim 13, further comprising one or more separation layer(s) between the hydrogel and either the conformable layer and/or the moisture regulation layer.

15. A dressing of claim 13, further comprising a film layer covering the moisture regulation layer.

16. A dressing of claim 13, wherein the conformable layer contains elastane.

17-20. (canceled)

21. The dressing of claim 1, further comprising a securing netting securing the hydrogel to the conformable layer.

22. The wound dressing of claim 1, wherein the amount of ionic silver is determined using Test 1.

23. The wound dressing of claim 1, wherein the amount of ionic silver is determined using Test 2.
